(19) United States
(12) Patent Application Publication
(10) Pub. No.: US 2025/0201399 A1
(43) Pub. Date: Jun. 19, 2025

(54) METHODS FOR CONTROL OF A SEQUENCING DEVICE

(71) Applicant: Life Technologies Corporation, Carlsbad, CA (US)

(72) Inventors: Jing Gao, San Jose, CA (US); Vidya Kudlingar, Foster City, CA (US); Dhirendrakumar Rai, Tracy, CA (US)

(21) Appl. No.: 18/968,214

(22) Filed: Dec. 4, 2024

Related U.S. Application Data
(63) Continuation of application No. 16/945,052, filed on Jul. 31, 2020, now abandoned.
(60) Provisional application No. 62/704,806, filed on May 29, 2020.
(60) Provisional application No. 62/889,109, filed on Aug. 20, 2019.

(51) Int. Cl. G16H 40/63; C12Q 1/6869; G06F 21/62; G16B 30/10; G16B 50/00; G16H 40/40; G16H 40/67

(52) U.S. Cl. CPC G16H 40/63; C12Q 1/6869; G06F 21/6218; G16B 30/10; G16B 50/00; G16H 40/40; G16H 40/67

(57) ABSTRACT

Methods and systems that use cloud-based resources and assay definition files for a local server system to control a sequencing device and process sequencing data resulting from a sequencing run for an assay are described. A method may include receiving, at a local server system, an assay definition file from a server of a cloud computing and storage system. The assay definition file may include code modules for configuring an assay. The code modules may be stored in a memory of the local server system. The server system may receive sequencing data from a sequencing device. The sequencing device may produce the sequencing data during a sequencing run performed for the assay. The server system may apply an analysis pipeline for the assay to the sequencing data. The analysis pipeline includes analysis steps executed in accordance with the code modules from the assay definition file to produce assay analysis results.

### CROSS-REFERENCE TO RELATED APPLICATIONS

This application is a Continuation of U.S. application Ser. No. 16/945,052 filed Jul. 31, 2020, which claims benefit under 35 U.S.C. § 119(e) of U.S. Provisional Application No. 62/889,109, filed Aug. 20, 2019, and U.S. Provisional Application No. 62/704,806, filed May 29, 2020. The entire contents of the aforementioned applications are incorporated by reference herein.

### FIELD

The present disclosure relates to control of a sequencing device for next generation sequencing (NGS) including digital delivery of modular software components containing assay workflows from a cloud-based computing and storage system resource.

### BACKGROUND

Increasingly, biological and medical research is turning to nucleic acid sequencing for enhancing biological studies and medicine. For example, biologists and zoologists are turning to sequencing to study the migration of animals, the evolution of species, and the origins of traits. The medical community is using sequencing for studying the origins of disease, sensitivity to medicines, and the origins of infection. As such, sequencing has wide applicability in practically every aspect of biology, therapeutics, diagnostics, forensics and research.

Nevertheless, the use of sequencing can be limited by assay availability, sequencing run time, preparation time, and cost. Additionally, quality sequencing has historically been an expensive process, thus limiting its practice.

### SUMMARY

Molecular pathology testing by a laboratory may be enhanced by a large selection of assays developed for a sequencing device, such as an NGS sequencing device. A server system configured to control the sequencing device may implement a modular software platform that supports rapid expansion of the molecular test menu, enabling rapid adoption of assays by laboratories. The assay contents and corresponding workflows may be delivered as modular software components from a cloud-based computing and storage system resource (e.g. Thermo Fisher Cloud, Thermo Fisher Scientific; Waltham, MA). The assay configuration content and corresponding workflows are delivered to the user's server system as modular software components in an assay definition file (ADF). The assay definition file supports backward compatibility of the workflow software modules and separation of the workflow software modules from the platform software of the server system.

The server system and modular software components may be configured to control multiple functional modes, including a research use only (RUO), or assay development (AD), mode and an in vitro diagnostics (IVD), or Dx, mode. The RUO, or AD, mode supports development and digital delivery of assays for research applications and third-party development of assays (RUO and AD used interchangeably). The IVD, or Dx, mode supports digital delivery of molecular diagnostic assays that have fulfilled local requirements for diagnostic applications (IVD and Dx used interchangeably). The multiple functional modes enable the same NGS sequencing device to be utilized for both RUO assays and IVD assays.

According to an exemplary embodiment, there is provided a method including the following steps: receiving, at a local server system, an assay definition file from a server of a cloud computing and storage system, wherein the assay definition file includes code modules for configuring an assay; storing the code modules in a memory of the local server system; receiving, at the local server system, sequencing data from a sequencing device, the sequencing data produced by the sequencing device during a sequencing run for the assay; and applying an analysis pipeline for the assay to the sequencing data, wherein the analysis pipeline includes analysis steps executed by a processor of the local server system in accordance with the code modules from the assay definition file to produce assay analysis results.

According to an exemplary embodiment, there is provided a local server system comprising a memory and a processor configured to execute instructions, which, when executed by the processor, cause the local server system to perform a method, comprising: receiving, at the local server system, an assay definition file from a server of a cloud computing and storage system, wherein the assay definition file includes code modules for configuring an assay; storing the code modules in the memory of the local server system; receiving, at the local server system, sequencing data from a sequencing device, the sequencing data produced by the sequencing device during a sequencing run for the assay; and applying an analysis pipeline for the assay to the sequencing data, wherein the analysis pipeline includes analysis steps executed by the processor of the local server system in accordance with the code modules from the assay definition file to produce assay analysis results.

### BRIEF DESCRIPTION OF THE DRAWINGS

The novel features are set forth with particularity in the appended claims. A better understanding of the features and advantages will be obtained by reference to the following detailed description that sets forth illustrative embodiments and the accompanying drawings of which:

FIG. 1 shows a schematic diagram of the server system components, in accordance with an embodiment.

FIG. 2 is a block diagram of the analysis pipeline, in accordance with an embodiment.

FIG. 3 is a schematic diagram of generating an assay definition file, in accordance with an embodiment.

FIG. 4 is a schematic diagram of an example of the assay definition file packaging.

FIG. 5 is an illustration of an example of a sequencing instrument.

FIG. 6 is an illustration of an example of an instrument deck of the sequencing instrument of FIG. 5.

FIG. 7 is a diagram representing an example of the workflow of the sequencing instrument.

FIG. 8 is an illustration of an example of a sequencing chip.

FIG. 9 is a block diagram of an example of processing the sequencing data from multiple lanes of the sequencing chip.

### DETAILED DESCRIPTION

As used herein, DNA (deoxyribonucleic acid) may be referred to as a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. In various embodiments, “nucleic acid sequencing data,” “nucleic acid sequencing information,” “nucleic acid sequence,” “genomic sequence,” “genetic sequence,” or “fragment sequence,” “nucleic acid sequence read” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, for example 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “variants,” “genomic variants” or “genome variants” denote a single or a grouping of sequences (in DNA or RNA) that have undergone changes as referenced against a particular species or sub-populations within a particular species due to mutations, recombination/crossover or genetic drift. Examples of types of genomic variants include, but are not limited to: single nucleotide polymorphisms (SNPs), copy number variations (CNVs), insertions/deletions (indels), single nucleotide variant (SNVs), multiple nucleotide variants (MNVs), inversions, etc.

In various embodiments, genomic variants can be detected using a nucleic acid sequencing system and/or analysis of sequencing data. The sequencing workflow can begin with the test sample being sheared or digested into hundreds, thousands or millions of smaller fragments which are sequenced on a nucleic acid sequencer to provide hundreds, thousands or millions of sequence reads, such as nucleic acid sequence reads. Each read can then be mapped to a reference or target genome, and in the case of mate-pair fragments, the reads can be paired thereby allowing interrogation of repetitive regions of the genome. The results of mapping and pairing can be used as input for various standalone or integrated genome variant (for example, SNP, CNV, Indel, inversion, etc.) analysis tools.

The phrase “sample genome” can denote a whole or partial genome of an organism.

As used herein, a “targeted panel” refers to a set of target-specific primers that are designed for selective amplification of target gene sequences in a sample. In some embodiments, following selective amplification of at least one target sequence, the workflow further includes nucleic acid sequencing of the amplified target sequence.

As used herein, “target sequence” or “target gene sequence” and its derivatives, refers to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In various embodiments, target nucleic acids generated by the amplification of multiple target-specific sequences from a population of nucleic acid molecules can be sequenced. In some embodiments, the amplification can include hybridizing one or more target-specific primer pairs to the target sequence, extending a first primer of the primer pair, denaturing the extended first primer product from the population of nucleic acid molecules, hybridizing to the extended first primer product the second primer of the primer pair, extending the second primer to form a double stranded product, and digesting the target-specific primer pair away from the double stranded product to generate a plurality of amplified target sequences. In some embodiments, the amplified target sequences can be ligated to one or more adapters. In some embodiments, the adapters can include one or more nucleotide barcodes or tagging sequences. In some embodiments, the amplified target sequences once ligated to an adapter can undergo a nick translation reaction and/or further amplification to generate a library of adapter-ligated amplified target sequences.

In various embodiments, the method of performing multiplex PCR amplification includes contacting a plurality of target-specific primer pairs having a forward and reverse primer, with a population of target sequences to form a plurality of template/primer duplexes; adding a DNA polymerase and a mixture of dNTPs to the plurality of template/primer duplexes for sufficient time and at sufficient temperature to extend either (or both) the forward or reverse primer in each target-specific primer pair via template-dependent synthesis thereby generating a plurality of extended primer product/template duplexes; denaturing the extended primer product/template duplexes; annealing to the extended primer product the complementary primer from the target-specific primer pair; and extending the annealed primer in the presence of a DNA polymerase and dNTPs to form a plurality of target-specific double-stranded nucleic acid molecules.

As used herein, the term “templating” refers to a process of generating two or more, or a plurality or population, of substantially identical polynucleotides, or of generating a substantially monoclonal population of nucleic acids, that can be used as templates in nucleic acid analysis methods, including, for example, nucleic acid sequencing, such as sequencing by synthesis, of the polynucleotides. The polynucleotides generated in a templating process are typically referred to as nucleic acid templates.

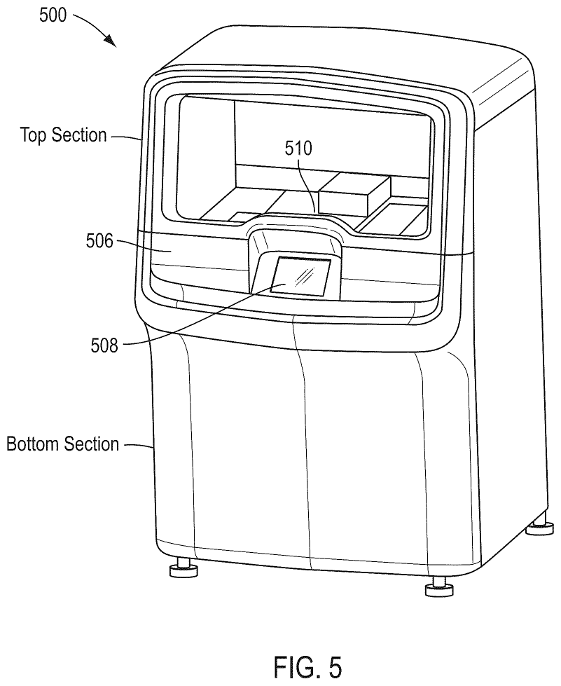

In some embodiments, a nucleic acid sequencing instrument maybe interfaced with a server system for control of various components of the sequencing instrument and processing of data output from sequencing runs on the sequencing instrument. The server system software may include a web application, databases and analysis pipeline and support connections from a sequencing instrument (FIG. 5). The server system software may provide the following major functionalities and application program interfaces (APIs):
1. APIs for user authentication, reagent tracking, run information and run tracking/logging. Supported instruments may include the sequencing instrument and extraction instrument.
2. APIs for a LIMS (Laboratory Information Management System) for creation of samples, libraries, plan run and retrieve the run status of the plan.
3. Support for management of samples and run data.
4. Support for assay configuration and execution of the analysis pipeline for data analysis and reporting.
5. Interface to a software update server for software updates and maintenance.
6. Supports configuration to connect to an annotation and reporting system, such as Ion Reporter from Thermo Fisher Scientific, deployed in a cloud-based system or a local system, and establishes secure and authenticated connection with the cloud-based system to transfer mapped or unmapped BAM files.
7. Supports configuration to connect to a resource system in a cloud computing environment, such as the Thermo Fisher Cloud, and establishes secure and authenticated connection with the cloud resource system to download software and system contents and to send telemetry data.

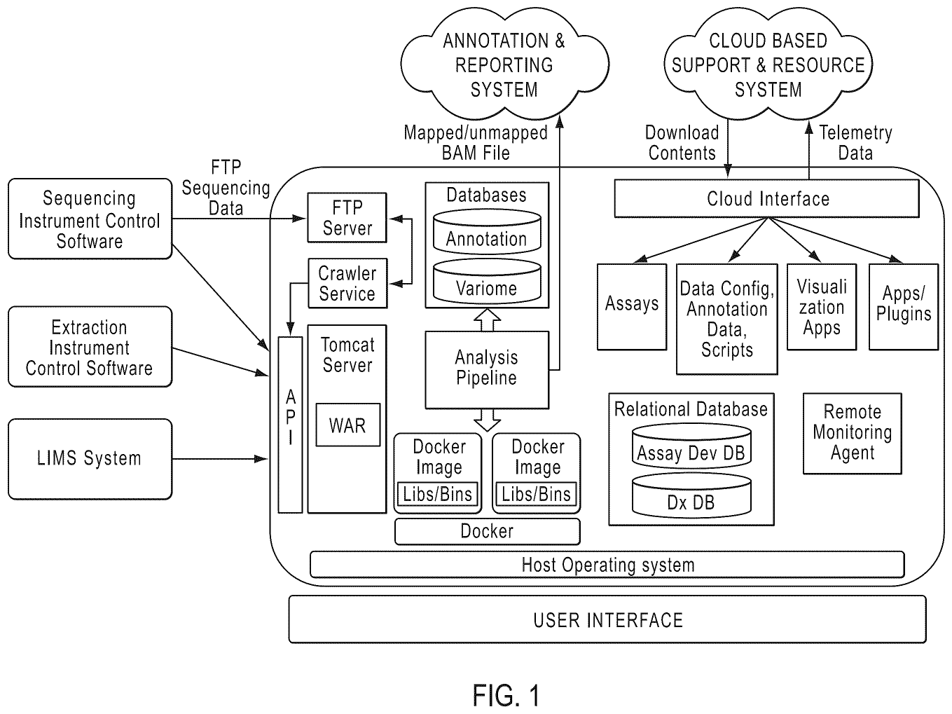

FIG. 1 shows a schematic diagram of the server system components. In some embodiments, the basic software architecture may comprise a web interface, remote monitoring agent, databases, APIs to the instruments, analysis pipeline, containerization of the analysis pipeline (using Docker, for example), connectivity to an annotations and reporting system (e.g. Ion Reporter from Thermo Fisher Scientific) and a cloud-based support and resource system (e.g. Thermo Fisher Cloud). The cloud-based support and resource system, or cloud-based resource system, may be implemented in a cloud computing and storage system. The cloud-based support and resource system stores content including assay definition files. A server of the cloud computing and storage system may download contents, such as assay definition files, to the local server system. The cloud-based support and resource system may receive telemetry data from the local server system. Server system, local server system and user's server system are used interchangeably herein.

In some embodiments, a user interface (UI) may be implemented via web application software. The UI may provide sample management pages. The sample management UI pages allow the user to enter sample information into the system. Sample information includes unique sample identifier (ID), sample name and sample preparation reagent tracking information. Validation logic is built into the sample management flow that locks the sample preparation step to the pre-defined assay workflow. The UI may provide assay management pages. Assay management UI pages allow the user to view assays, and create assays. The assays lock the workflows to pre-defined parameters for each step of the process. Validation logic may be built in to ensure the assay configuration. The UI may provide run plan and monitor pages. The run plan and monitor UI pages allow the user to plan for a run and monitor the run in progress. The UI may provide output data pages. The output data UI pages allow the user to view the analysis results along with quality control (QC) metric evaluation, log and audit trail of the results generated. The UI may provide configuration pages. The configuration UI pages allow users to view and configure the system.

In some embodiments, application programming interfaces (APIs) may be provided through a Java platform. For example, the Java platform may include a Tomcat server that may be used to build a Web ARchive (WAR) file for web-based applications.

Code modules for various steps of the analysis pipeline may be referred to as actors in the context of a Kepler workflow engine. For example, a code module for an analysis step may implemented by Java program binary code included in an actor jar. A Kepler workflow engine defines processing components of a workflow as “actors” and chains the steps for execution by a processor of the algorithm or analysis pipeline. (www.kepler-project.org). For example, a Kepler workflow engine may be used to configure the workflow of the analysis pipeline in FIG. 1.

The server system may include one or more databases. For example, the server system may include a relational database for storing sample data, run data and system/user configuration. The relational database may include two separate databases: assay development database and Dx database. The assay development database may store sample data, run data and system/user configuration for RUO, or assay development, mode of operation. The Dx database may store sample data, run data and system/user configuration for the IVD, or Dx, mode of operation.

The server system may include an annotations database, AnnotationDB, for storing annotation source data. For example, the annotations database may be implemented as NoSQL, or non-relational, database, e.g. a MongoDB database. Each annotation source may be stored as a JSON (JavaScript Object Notation) string with meta information indicating source name and version. Each annotation source may contain a list of annotations keyed to annotation IDs. The server system may include a variome database, VariomeDB, for storing variant information. For example, the variome database may be implemented as a NoSQL, or non-relational, database, e.g. a MongoDB database. The VariomeDB may store a collection of variant call results on a particular sample. For example, a JSON formatted record may contain meta information for identifying the sample.

For example, the AnnotationDB database may store one or more of the following annotation sources:
1. RefGene Model: hg19_refgene_63, version 63
2. RefGene Functional Canonical Transcripts Scores: hg19_refgeneScores_4, version 4
3. dbSNP: dbsnp_138, version 138
4. Canonical RefSeq Transcripts: hg19_refgene_63, version 63
5. 5000Exomes: hg_esp6500_1, version 1
6. ClinVar: clinvar_1, version 1
7. DGV: dgv_20130723, version 20130723
8. OMIM: omim_03022014, version 03022014

Other annotation sources may be included. Other versions of the above annotation sources may be included. The annotation source may provide public annotation information content or proprietary annotation information content.

For each call in Variome database, and each annotation source may be queried for annotations matching the variant and matching annotations may be stored as key-value pairs in Variome database with the variant. Annotated variants may be included in a results file, e.g. an annotated VCF file, for the user. VCF files are tab-separated text files used for storing gene sequence variants. In some embodiments, the annotation methods for use with the present teachings may include one or more features described in U.S. Pat. Appl. Publ. No. 2016/0026753, published Jan. 28, 2016, incorporated by reference herein in its entirety.

In some embodiments, the server system may include an analysis pipeline to process sequencing data generated during a sequencing run for an assay performed by a sequencing instrument. The sequencer transfers sequencing data files and experiment log files to the server system memory, for example in raw .dat files, already processed .dat files producing block wise 1.wells files, and thumbnail data. The analysis pipeline accesses the data files from memory and starts data analysis for the run.

In some embodiments, a Docker container and Docker images may be used for packaging the analysis pipeline and operating system specific binaries. The Docker is a tool used to create, deploy and run applications by using containers. Containers enable an application with all the parts it needs, such as libraries and other dependencies, to be bundled as one package. This allows applications software to use the same Linux kernel as the host system. The Docker image files may be packaged with libraries and binaries needed by the analysis pipeline code. The Docker may be used to adapt an application or algorithm to a new or different version of an operating system (OS) to create a Docker image of the application that is compatible with the OS version.

In some embodiments, the server system may include a crawler service for data transfer from the sequencing instrument to the analysis pipeline. The crawler is an event based service that may be developed using JAVA NIO watcher API (application programming interface). NIO (Non-blocking I/O) is a collection of Java programming language APIs that offer features for intensive input/output (I/O) operations. The crawler may monitor the FTP directory configured for the sequencing instrument to transfer run data from the sequencing instrument to the analysis pipeline.

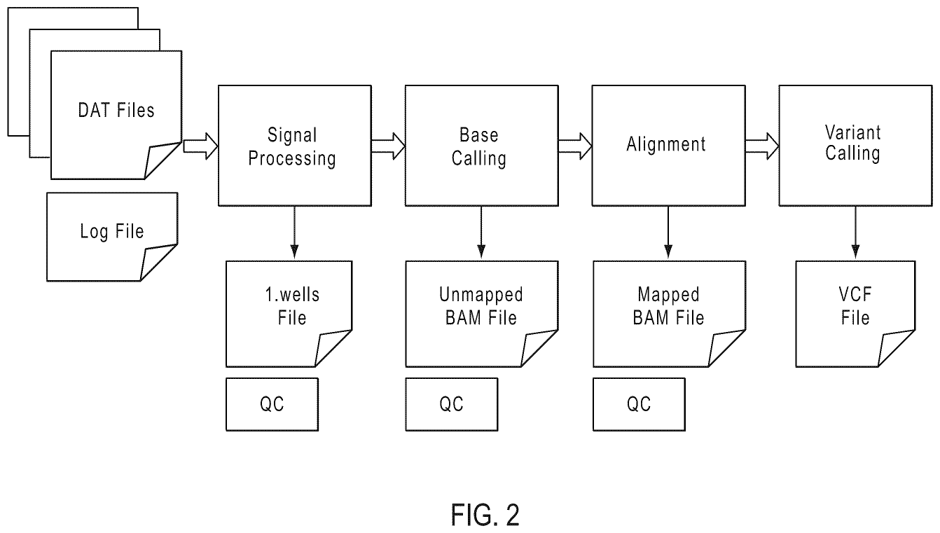

FIG. 2 is a block diagram of the analysis pipeline, in accordance with an embodiment. The sequencing instrument generates raw data files (DAT, or .dat, files) during a sequencing run for an assay. Signal processing may be applied to raw data to generate incorporation signal measurement data for files, such as the 1.wells files, which are transferred to the server FTP location along with the log information of the run. The signal processing step may derive background signals corresponding to wells. The background signals may be subtracted from the measured signals for the corresponding wells. The remaining signals may be fit by an incorporation signal model to estimate the incorporation at each nucleotide flow for each well. The output from the above signal processing is a signal measurement per well and per flow, that may be stored in a file, such as a 1.wells file.

In some embodiments, the base calling step may perform phase estimations, normalization, and runs a solver algorithm to identify best partial sequence fit and make base calls. The base sequences for the sequence reads are stored in unmapped BAM files. The base calling step may generate total number of reads, total number of bases and average read length as QC measures to indicate the base call quality. The base calls may be made by analyzing any suitable signal characteristics (e.g., signal amplitude or intensity). The signal processing and base calling for use with the present teachings may include one or more features described in U.S. Pat. Appl. Publ. No. 2013/0090860 published Apr. 11, 2013, U.S. Pat. Appl. Publ. No. 2014/0051584 published Feb. 20, 2014, and U.S. Pat. Appl. Publ. No. 2012/0109598 published May 3, 2012, each incorporated by reference herein in its entirety.

Once the base sequence for the sequence read is determined, the sequence reads may be provided to the alignment step, for example, in an unmapped BAM file. The alignment step maps the sequence reads to a reference genome to determine aligned sequence reads and associated mapping quality parameters. The alignment step may generate a percent of mappable reads as QC measure to indicate alignment quality. The alignment results may be stored in a mapped BAM file. Methods for aligning sequence reads for use with the present teachings may include one or more features described in U.S. Pat. Appl. Publ. No. 2012/0197623, published Aug. 2, 2012, incorporated by reference herein in its entirety.

The BAM file format structure is described in “Sequence Alignment/Map Format Specification,” Sep. 12, 2014 (www.github.com/samtools/hts-specs). As described herein, a “BAM file” refers to a file compatible with the BAM format. As described herein, an “unmapped” BAM file refers to a BAM file that does not contain aligned sequence read information and mapping quality parameters and a “mapped” BAM file refers to a BAM file that contains aligned sequence read information and mapping quality parameters.

In some embodiments the variant calling step may include detecting single-nucleotide polymorphisms (SNPs), insertions and deletions (InDels), multi-nucleotide polymorphisms (MNPs) and complex block substitution events. In various embodiments, a variant caller can be configured to communicate variants called for a sample genome as a *.vcf, *.gff, or *.hdf data file. The called variant information can be communicated using any file format as long as the called variant information can be parsed and/or extracted for analysis. The variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, and U.S. Pat. No. 9,953,130 issued Apr. 24, 2018, each of which is incorporated by reference herein in its entirety. In some embodiments, the variant calling step may be applied to molecular tagged nucleic acid sequence data. Variant detection methods for molecular tagged nucleic acid sequence data may include one or more features described in U.S. Pat. Appl. Publ. No. 2018/0336316, published Nov. 22, 2018, incorporated by reference herein in its entirety.

In some embodiments, the analysis pipeline may include a fusion analysis pipeline for fusion detection. Fusion detection methods may include one or more features described in U.S. Pat. Appl. Publ. No. 2016/0019340, published Jan. 21, 2016, incorporated by reference herein in its entirety. In some embodiments, the fusion analysis pipeline may be applied to molecular tagged nucleic acid sequence data. Fusion detection methods for molecular tagged nucleic acid sequence data may include one or more features described in U.S. Pat. Appl. Publ. No. 2019/0087539, published Mar. 21, 2019, incorporated by reference herein in its entirety.

In some embodiments, the analysis pipeline may include a copy number variants analysis pipeline for detection of copy number variations. Methods for detection of copy number variation may include one or more features described in U.S. Pat. Appl. Publ. No. 2014/0256571, published Sep. 11, 2014, U.S. Pat. Appl. Publ. No. 2012/0046877, published Feb. 23, 2012, and U.S. Pat. Appl. Publ. No. US2016/0103957, published Apr. 14, 2016, each of which is incorporated by reference herein in its entirety.

In some embodiments, the server system software may support an encapsulated assay configuration that includes assay name, assay type, panel, hotspot file if any, reference name, control names if any, quality control QC thresholds, assay description if any, data analysis parameters and values, instrument run script names and other configurations that define the assay. The entire set of the information is called an assay definition. The assay configuration content and corresponding workflows may be delivered to the user as modular software components in an assay definition file (ADF). The server system software may import an assay definition file that contains the assay configuration. The import process may be initiated by zip file import which includes an encrypted Debian file and triggers an installation process. The user interface may provide a page for the user to select an ADF for import. An application store in the cloud-based support and resource system may store ADFs supporting various assays, panels and workflows available for selection by the user for download to the user's local server system.

An assay definition file (ADF) is an encapsulated file that defines configurations for the molecular test or assay, including assay name, technology platform configuration (for example, next generation sequencing (NGS), chip type, chemistry type), workflow steps (sample prep, instrument scripts, analytics, reporting), analysis algorithms, regulatory labels (for example, research use only (RUO), in vitro diagnostics (IVD), Central Europe in vitro diagnostics (CE-IVD, internal use only (IUO), etc.), targeted markers (panel), reference genome version, consumables, controls, QC thresholds, reporting genes and variants. The ADFs provide a modular approach to building assay capabilities for the local sequencing instrument. The assay software may be provided by the ADF separately from the platform software of the sequencing instrument.

The advantages of using the ADF for assay configuration include the following:
Encapsulation of the assay workflow and analysis
Single click for installation
No revalidation required after software update for assay configuration because of the modular structure of the software by the Docker implementation allowing separation from the platform software
Multi-tiered encryption for secure delivery
Streamlined support of assay configurations for original equipment manufacturers (OEM)
Streamlined customization of reporting
Support of regional regulatory requirements
Plug-n-play format supports technology agnostic workflows
Enables rapid expansion of molecular test menu and assay adoption by laboratories

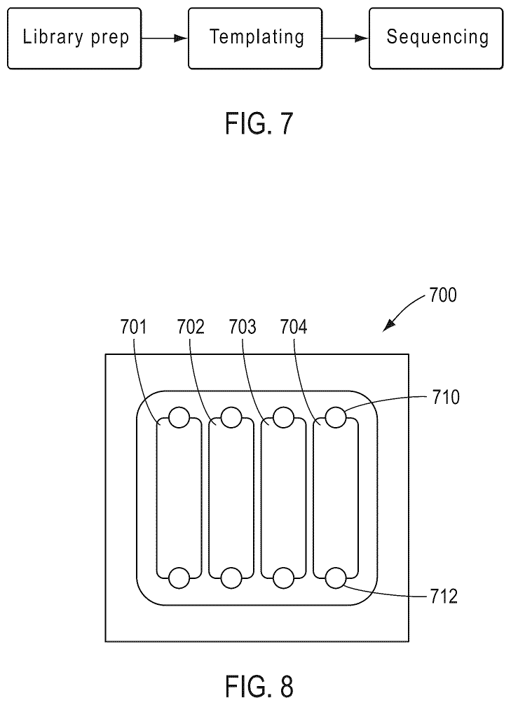

In some embodiments, the assay definition file (ADF) may include software code modules for one or more of the following steps 1) library preparation; 2) templating; 3) sequencing; 4) analysis; 5) variant interpretation; and 6) report generation. For the workflow steps of library preparation and templating (FIG. 7), the ADF may include scripts for preparing libraries, templating and enrichment of templated beads. For the workflow steps of sequencing and analysis the ADF may include Docker image packages of algorithm binary code and parameters for the analysis pipeline described with respect to FIG. 2. For the workflow step of variant interpretation, the ADF may include a list of annotation sources that may be used for analyzing and annotating variants. For the workflow step of report generation, the ADF may include report templates and image files for use when a generating a report.

The ADF may include for the instrument scripts for control of workflow steps on the sequencing instrument. For example, scripts may include parameters controlling the amount of pipetting and robotic control. The instrument scripts may be customized for the particular assay.

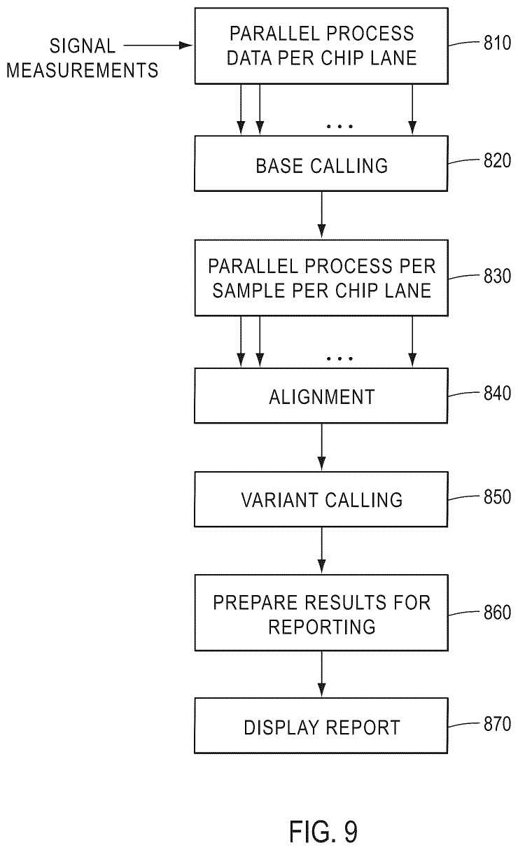

For example, for the sequencing and analysis steps, the ADF may include a Docker image of the end to end analysis pipeline. The Docker image may include OS specific libraries and binaries for the algorithms each step of analysis pipeline. The algorithm binaries may include steps of the analysis pipeline including signal processing, base calling, alignment and variant calling, such as those described with respect to FIG. 2 and FIG. 9. In another example, the ADF Debian file may package certain code modules for a particular assay, such as code modules for signal processing, base calling and RNACounts.

The ADF may include scripts for configuration of reagent kits. These scripts support calculation of the consumables needed for a sequencing run, as further described below with respect to Table 1. The configurations scripts included in the ADF may include one or more of the following:
Barcode set and chip
Library kit and consumables, including capability to associate sample control configuration, (e.g. sample inline control) and its QC parameters
Templating kit and consumables, including capability to associate internal controls and QC parameters
Sequencing kit, including capability to associate internal controls and QC parameters

The ADF may include one or more reference genome files. Examples of reference genomes include hg19 and GRCH38. The reference genome file may be packaged in the main ADF with the workflow information. Alternatively, the reference genome file may be packaged in a separate ADF that is supplementary to the main ADF.

The ADF may include code modules for workflows of fusion panels and fusion target region panels. The ADF may include fusion target region reference files and hotspot files for analysis.

The ADF may include assay parameters at various points of the workflow that may be configured by the user. The configurable parameters may be displayed in the user interface for adjustment by the user. New parameters may be added at any actor level. The configurable parameters may be passed to the analysis pipeline. Input formats for the configurable assay parameters may include one or more single string text, Boolean, multiline text, floating point, radio buttons, drop downs, and file uploads. For example, the file uploads may use file formats such as .properties and .json.

The ADF may include QC parameters used for quality control and assay performance thresholds at various points in the workflow. For example, types of QC parameters include run QC parameters, sample QC parameters, internal control QC parameters and assay specific QC parameters. A QC parameter may be defined by one or more of a data type (e.g. integer, floating point), lower bound, upper bound and default value.

The ADF may include specified data tab columns for results presentation that are selected from the database for a given assay. The selected data tab columns support configuration of the user interface display of results and the columns to be included in the PDF reports for the assay. The ADF may include image files for results presentation for a given assay. The ADF may include support for multiple languages for the PDF reports. The ADF may include a download file list for any files to be generated by the analysis pipeline for a given assay. The file list for the sample or run may be displayed at the user interface. The ADF may include a gene list. The gene list may be used to display the known list of genes for a given cancer type at the user interface and in a PDF report.

The ADF may include a set of plugins to be used for a given assay. The ADF may specify a set of plugins and their versions. If the ADF does not specify a version of a plugin, the latest version of the plugin installed on the server system may be used for the given assay.

The ADF may include a new workflow template to support custom assay creation. The new workflow template may include a set of assay chevron steps. Parameters for the steps may be displayed.

The ADF may include a list of annotation sources and sets to support the configuration of new annotation sets. The ADF may include filter chains to be applied to variants detected by the analysis pipeline of a given assay. The ADF may include rulesets for annotation of variants.

The ADFs can be configured to support a number of different types of assays. Examples include, but are not limited to, oncology related assays (e.g., Oncomine assays from Thermo Fisher Scientific), immuno-oncology related assays (e.g., T-cell receptor (TCR), microsatellite instability (MSI) and tumor mutation load (TML)), infectious diseases related assays (e.g. microbiome), reproductive health related assays and exome related assays. The ADF can also be configured for a custom assay.

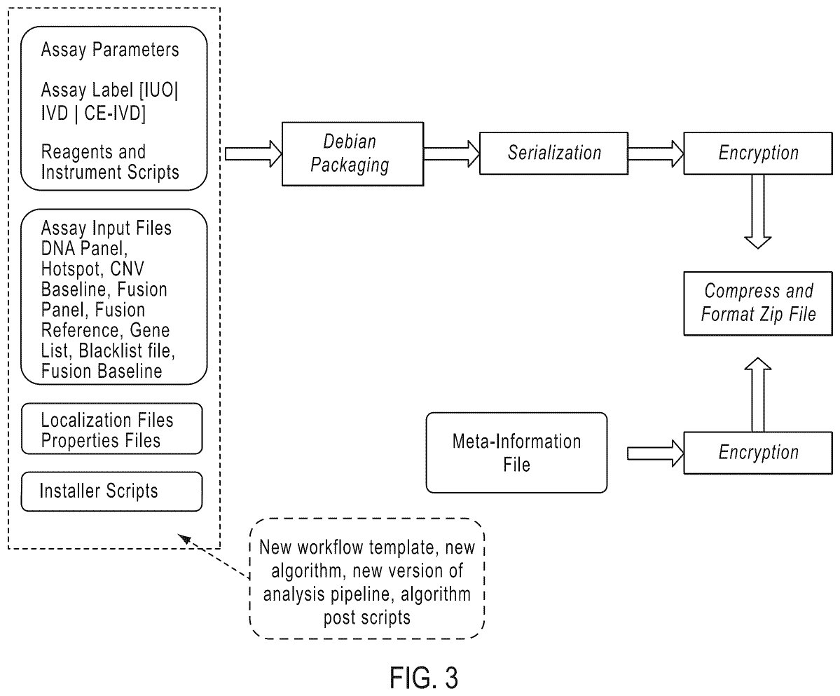

FIG. 3 is a schematic diagram of generating an assay definition file, in accordance with an embodiment. The assay definition may be generated by build.sh, debscripts and makedeb.sh that initiate file copying and database population of assay information to form a Debian file. The assay definition content may include assay parameters, BED files (Browser Extensible Data file—BED file—defines chromosome positions or regions), panel files, gene lists, hotspot files (a BED or a VCF file that defines regions in the gene that typically contain variants), and seed data containing allowable reagents. The assay definition content may contain localized versions of an assay name, description and report messages that support assay information display in different languages. The assay definition file may support the packaging of a new analysis pipeline. The ADF may include an optional post processing script which may be executed for variant calling, fusion calling and CNV calling based on the type of assay. The ADF may include an optional Docker container image of updates to the binaries for a specific analysis pipeline. The Docker container image may be packaged with the ADF to ensure that platform changes such as operating system or third-party library do not impact the results of the assays or functioning of the system.

The Debian file may be serialized to prevent unauthorized modifications. The serialized assay definition may be further encrypted using Advanced Encryption Standard (AES), a symmetric-key algorithm. A text file containing assay meta-information may also be encrypted using AES and the same encryption key. The encrypted assay definition file, together with the encrypted meta-information file may be compressed into zip format. Other encryption formats may also be applied to the serialized assay definition information. For example, the meta-information may include one or more of the following:
Analysis pipeline version,
Reference genome path for the reference genome file location,
Assay unique name—the assay's internal name for checking the unique occurrence in the system,
Docker image name—to be used for launching analysis and installing assay dependent file references,
Any dependency package names needed for analysis pipeline launch.

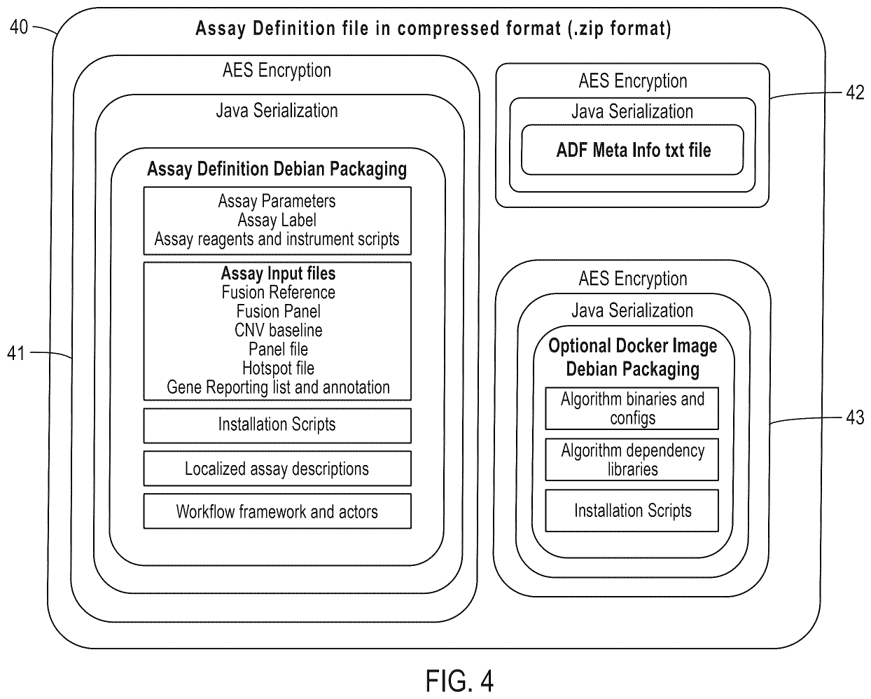

FIG. 4 is a schematic diagram of an example of the assay definition file packaging. The compressed assay definition file in zipped format 40 may include the serialized and encrypted assay definition Debian packaging 41, the serialized and encrypted meta-information text file 42, and serialized and encrypted optional Docker image Debian packaging 43. The server system may decrypt both the meta-information text file 42 and the assay definition serialized file 41 before installing the assay definition Debian file.

The server system and modular software components may be configured to control multiple functional modes, including an RUO, or AD, mode and an IVD, or Dx, mode. Referring to FIG. 1, the Tomcat Server may be configured to include a Web ARchive (WAR) file for the RUO mode and a WAR file for the IVD mode. The server system may be configured to include a RUO variome database for the variants detected by RUO assays and an IVD variome database for the variants detected by IVD assays. The server system may be configured to include separate analysis pipelines and associated Kepler workflow engines for the RUO mode and the IVD mode. The RUO Docker image files for the RUO assays may be configured as separate files from the IVD Docker image files for the IVD assays. The relational databases may be configured to have separate databases: an assay development (AD) database for the RUO mode and a Dx database for the IVD mode. A server system that initially supports only a RUO mode may be configured to support RUO and IVD modes by a software update.

ADFs may be generated separately for RUO mode assays and IVD mode assays. The RUO mode ADFs may include assay definitions for assays used in research. The RUO mode ADFs may be developed by a third party. The IVD mode ADFs include assay definitions for assays compliant with regional regulatory requirements for diagnostic use.

FIG. 5 includes an illustration of an example instrument 500 incorporating a three-axis pipetting robot. In an example, the instrument 500 can be a sequencer incorporating a sample prep preparation platform. For example, the instrument 500 can include an upper portion and a lower portion. The upper portion can include a door 506 to access a deck 510 on which samples, reagent containers, and other consumables are placed. The lower portion can include a cabinet for storing additional reagent solutions and other parts of the instrument 500. In addition, the instrument can include a user interface, such as a touchscreen display 508.

In a particular example, the instrument 500 can be a sequencing instrument (sequencing instrument, sequencing device and sequencer used interchangeably). In some embodiments, the sequencing instrument includes a top section, a display screen and a bottom section. In some embodiments, the top section may include a deck supporting components of the sequencing instrument and consumables, including a templating section, a sequencing chip and reagent strip tubes and carriers. In some embodiments, the bottom section may house reagent bottles containing reagents used for sequencing and a waste container.

In some embodiments, a camera mounted in a cabinet of the top section of the instrument is oriented towards the deck to monitor what items are in place in preparation for a sequencing run. The camera may acquire images at time intervals. For example, images may be acquired at 3-4 second intervals or any suitable interval. A processor analyses images to detect the completion of a task by the user. The processor may provide feedback and instructions for the next task in the preparation via the display screen. The display screen may present graphical representations of the instrument components and consumables in order to illustrate instructions for the user.

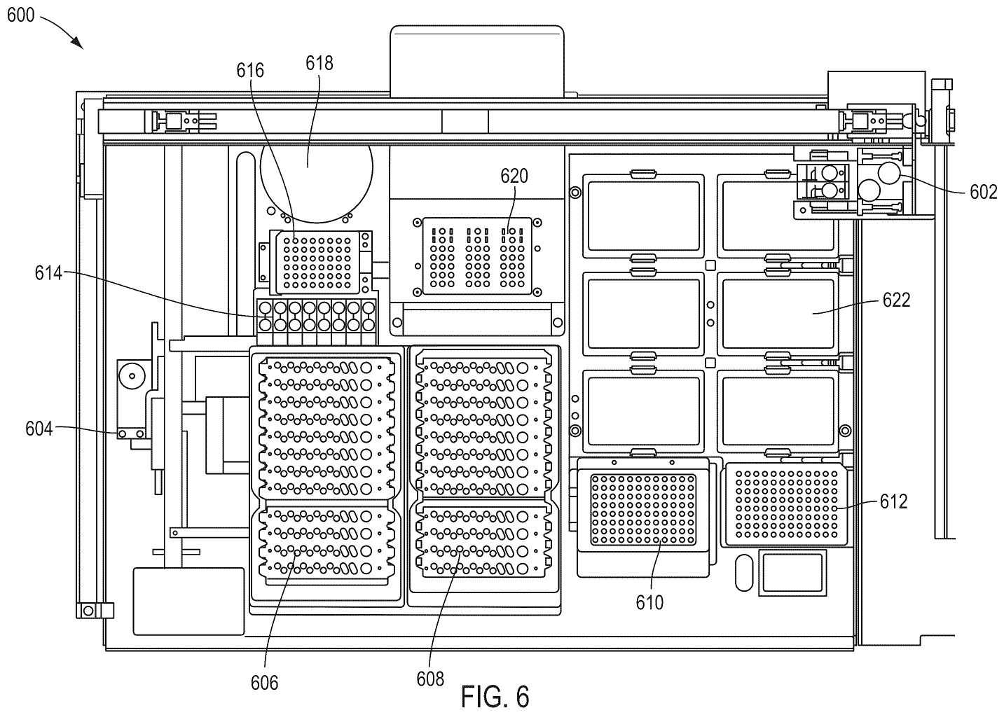

An example instrument deck 510 is illustrated in FIG. 6 as instrument deck 600. The instrument deck 600 is housed in the top section of the instrument in the view of the camera or cameras. The sample preparation deck may include a plurality of locations configured to receive reagent strips, supplies, a sequencing chip, and other consumables. As used herein, consumables are components used by the instrument that are replaced periodically as they are used. For example, consumables include reagent and solution strips or containers, pipette tips, microwell arrays, and flowcells and associated sensors, among other disposable components not part of the permanent components of the instrument.

In an example, the instrument deck system 600 includes a pipetting robot 602 that accesses various reagent strips and containers, pipette tips, microwell arrays, and other consumables to implement a test. Further, the system can include mechanisms 604 for carrying out testing. Example mechanisms 604 include mechanical conveyors or slides and fluidic systems.

In an example, the instrument deck 600 includes trays 606 or 608 to receive solution or reagent strips of a particular configuration. In an example of a sequencing instrument, the tray 606 can be used for library and template solutions in appropriately configured strips, and the tray 608 can receive library and template reagents in the appropriate configuration.

Further, the instrument can be configured to receive sequencing chips including microwell arrays 610 and 612 at particular locations on the deck. For example, a sample can be supplied in an array of microwells of a sequencing chip 612. In another example, the system can be configured to receive additional reagents 614 in a different strip configuration. In another example, reagent solutions can be provided in an array 616. In a further example, container arrays 620 can be provided in conjunction with instrumentation, such as a thermocycler. Further, the system can include other instrumentation, such as a centrifuge, that may be supplied with consumables, such as tubes. Further, trays can be provided to receive pipetting tips 622.

The appropriate provisioning of consumables in each of these locations can be monitored by a vision system including one or more cameras. The deck may be provided with one or more cameras to track provisioning and securing of reagents and other consumables. The user can be prompted through the user interface when a reagent is missing that is to be utilized to perform one plan or when a reagent consumable is present in a used state.

FIG. 7 is a diagram representing the workflow of the sequencing instrument. The top level steps include library preparation, templating and sequencing.

The sequencing instrument components may include a sequencing chip (interchangeably, microchip, chip or sensor device) including a microwell array, in fluid communication with a sensor array, and a flowcell having multiple lanes. FIG. 8 is an illustration of an example of a sequencing chip 700 having four lanes 701, 702, 703 and 704. Each lane is individually accessed by a respective fluid inlet 710 and fluid outlet 712. Alternatively, the sensor device 700 can include less than four lanes or more than four lanes. For example, the sensor device 700 can include between 1 and 10 lanes, such as between 2 and 8 lanes, or 4 to 6 lanes. The lanes can be fluidically isolated from each other. As such, the lanes can be used at separate times, concurrently, or simultaneously, depending upon aspects of a run plan.

It is advantageous to optimize use of the lanes of the sequencing chip for multiple assays. A given lane may accommodate more than one sample. In some embodiments, the server system software may provide for optimization of chip usage by applying on or more of the following rules:
Maximum number of assays allowed to be included in single plan run is equal to number of available chip lanes. This rule is applicable to both new and used chip.
   The maximum number of assays allowed in the single plan run may be adjusted depending on the number of lanes required by assay. Rules to determine the number of lanes may include the following:
      One Assay per lane
      If Assay's minimum number of reads per sample is more than the lane capacity, calculate the number of lanes needed, i.e. (minimum number of reads/lane capacity) e.g. 2000000/1300000=1.54 lanes, round up to 2, so assay requires 2 lanes
The combined pool size of the selected assay(s) may not exceed 8
   The combined pool size=sum (pool size of each assay)
   For AmpliSeq panels (Thermo Fisher Scientific), the pool size of AmpliSeq assay=sum (number of DNA pools, number of RNA pools)
   For AmpliSeq HD panels (Thermo Fisher Scientific), the pool size for AmpliSeq HD assay=number of TNA pools
The rules below may be applied for PCR profiles
   The number of distinct PCR profiles (thermo cycling) in a single plan run may not exceed 2
   For DNA and Fusions assays, the DNA samples and Fusions samples must be assigned to separate zones. This rule restricts the number of PCR profiles supported in a single plan run.
   TNA, DNA and Fusions assays can be run in a single plan. In this case TNA and RNA can go in the same zone if PCR profile for TNA and RNA is same. DNA may be in separate zone.
      The PCR profile is defined per assay.
      The PCR profile is an assay attribute stored in the database when saving an assay
      For factory shipped assays, PCR profile is pre-seeded
      For custom assays, the user may edit PCR profile during assay creation, which will be detailed in assay creation user story
The assays in a single plan run can have same or different analysis pipeline versions.
The assays in the single plan run can be of same or different application types (DNA only, RNA only, DNA+RNA, etc.).
The number of flows for all the assays in the single plan run need not be same. The highest number of flows will be used for the run. The analysis pipeline should analyze only the data for the number of flows configured in the assay. Setting a flow-limit parameter corresponding to the assay may limit the signal processing to the number of flows configured in the assay.
The assays in a single plan run can have different templating sizes.

In some embodiments, the software may be configured to show a warning message if chip type or capacity does not match with the plan in progress. For the example scenarios below, a confirmation dialogue with warning message can be displayed to the user. User's confirmation choice may be maintained, and the rest of the validation may happen based on the user's choice of considering a new chip or the on deck chip.
Selected Assay chip type does not match with the one on the deck, show a confirmation dialogue with warning message “The chip type on the deck does not match with the selected Assay”, Do you want consider new chip, click on Yes to consider new chip or click on cancel to use deck chip? with Yes and Cancel option.
Number of selected assays are more than the available lane capacity, show a confirmation message “The Chip on the deck have only N lanes available which can process N number of assays only”, Do you want to consider new chip?
One Assay Selected but number of reads per sample exceeds the available lane capacity, show a confirmation message “The selected assay exceeds the available lane capacity of the chip, so minimum reads per sample cannot be achieved”, Do you want to consider new chip?
   If Yes switch to new chip validation
   If No allow user to continue with his selected option
On Click on Next, software must assign lanes for each selected assay. Lane allocation rules may be as follows:
   One Assay per lane
   If Assay's min no of reads per sample is more than the lane capacity, calculate the lanes needed, i.e. (min no of reads/lane capacity) e.g. 2000000/1300000=1.54 lanes, round up to 2 so assay requires 2 lanes

The chip lane assignment rules may include the following:
Number of lanes assigned to an assay=Upper ceiling ((number of selected samples+controls)×Min reads per sample/reads per lane)
If multiple lanes are assigned to an assay, the assigned lanes must be consecutive. On samples page, after final number of lanes needed for an assay is determined, software must readjust lane assignment to have consecutive lane assignment.

FIG. 9 is an example of a block diagram for processing the sequencing data from multiple lanes of the sequencing chip. Preprocessing may prepare the analysis corresponding to each chip lane in accordance with the assay assigned to the lane. For example, the server software may create data structures such as pipeline folder structure for the assays corresponding to the individual lanes and a folder structure for each sample in each lane. Signal measurements resulting from signal processing, for example from a 1.wells file, as described with respect to FIG. 2, may be input to the parallel process block 810. The base calling step 820 may be applied to the plurality of signal measurements corresponding to each lane to determine the base sequences of a plurality of sequence reads for the lane. In step 830, the sequence reads per sample per lane are provided to the alignment step 840. The sequence reads may be provided to the alignment step, for example, in unmapped BAM files per sample per lane. The alignment step 830 maps the sequence reads to a reference genome. The mapped reads per sample per lane may be stored in mapped BAM files corresponding to the sample and lane. The variant calling step 850 may be applied in accordance with the assay type to the mapped reads corresponding to the sample and the lane. The base calling step 820, alignment step 840 and variant calling step 850 are described with respect to FIG. 2. A Kepler workflow engine may be applied to control the processing flow of one or more of the steps of FIG. 9. When the variant calling step 850 is complete for the samples and lanes, the results may be prepared for reporting at step 860. For example, the results may be used to populate PDF files and generate image files specific for the particular assay. At step 870, the results may be displayed to the user or provided in a PDF file.

In some embodiments, the server software may calculate the consumables needed for a sequencing run. Table 1 lists examples of consumables calculations.

TABLE 1

| | Consumable | | |
|---|---|---|---|
| Category | Name | Quantity | Comments |
| Bar-code | <Barcode | Quantity | 1. Should display the barcode plate loaded on deck if |
| Kit | Plate Name | will | the available barcodes on the loaded deck is equal to |
| | associated | always | or greater than the barcodes needed for the run. |
| | with the | be 1 | In this case Status is On Instrument |
| | Assay>-# | | |
| | Barcodes | | |
| | Needed | | |
| Library Kit | Library | Total | Status will always be Need New |
| | Reagents | number | |
| | Strips of | of primer | |
| | Library kit | pool | |
| | associated | tubes for | |
| | with the | the run | |
| | Assay | | |
| Library Kit | Library | Total | Status will always be Need New |
| | Solutions | number | |
| | Strips of | of primer | |
| | Library kit | pool | |
| | associated | tubes for | |
| | with the | the plan | |
| | Assay | run | |
| Templating | Templating | Total No | Status will always be Need New |
| Kit | Reagents | of lanes | |
| | Strips of | for the | |
| | Template kit | Run | |
| | associated | | |
| | with the | | |
| | Assay | | |
| Templating | Templating | Total No | Status will always be Need New |
| Kit | Solutions | of lanes | |
| | Strips of | for the | |
| | Template kit | Run | |
| | associated | | |
| | with the | | |
| | Assay | | |
| Templating | Pipette tips | <#> | Python script is available and getplannedrunbyid's |
| Kit | | calculated | deckconfig array json is the input for the script. |
| | | from | |
| | | the script | |
| Templating | PCR Plates | 3 | |
| Kit | | | |
| Sequencing | Sequencing | Quantity | 1. If On deck Chip have the available lanes |
| | Chip of Chip | will | needed for the Run then status will be On Instrument |
| | kit associated | always | 2. If New chip is needed for the run the status will be |
| | with the | be 1 | Need New |
| | Assay | | |
| Sequencing | Couplers | Quantity | Same status as Chip |
| | | will | |
| | | always | |
| | | be 1 | |
| Sequencing | Wash Bottles | Quantity | Same status as Chip |
| | | will | |
| | | always | |
| | | be 1 | |
| Sequencing | Cleaning | Quantity | Same status as Chip |
| | Solution | will | |
| | | always | |
| | | be 1 | |
| Sequencing | Nucleotides | Quantity | Same status as Chip |
| | Cartridge | will | |
| | | always | |
| | | be 1 | |

According to an exemplary embodiment, there is provided a method including the following steps: receiving, at a local server system, an assay definition file from a server of a cloud computing and storage system, wherein the assay definition file includes code modules for configuring an assay; storing the code modules in a memory of the local server system; receiving, at the local server system, sequencing data from a sequencing device, the sequencing data produced by the sequencing device during a sequencing run for the assay; and applying an analysis pipeline for the assay to the sequencing data, wherein the analysis pipeline includes analysis steps executed by a processor of the local server system in accordance with the code modules from the assay definition file to produce assay analysis results. The code modules for the analysis pipeline may include a code module for a base calling step, the base calling step producing sequence reads. The code modules for the analysis pipeline include a code module for an alignment step, the alignment step producing aligned sequence reads. The code modules for the analysis pipeline include a code module for a variant calling step, the variant calling step applied to the aligned sequence reads to produce variant call results. The method may further comprise storing the variant call results in a variome database of the local server system. The method may further comprise displaying the assay analysis results, wherein the display includes an image file for results presentation for the assay. The assay definition file may include the image file for the results presentation for the assay. The assay definition file may include a reference genome file. The assay definition file may include a list of annotation sources. The analysis pipeline may be applied in parallel to the sequencing data corresponding to multiple lanes of a sequencing chip installed in the sequencing device. Each lane of the multiple lanes may correspond to a respective assay, wherein the step of applying an analysis pipeline applies the analysis steps for the respective assay to the sequencing data for the lane. The method may further comprise displaying a page at a user interface of the local server system to a user for selection of the assay definition file for import to the local server system from the cloud computing and storage system. The method may further comprise a plurality of assay definition files, wherein the plurality of assay definition files includes a research use only (RUO) mode assay definition file and an in vitro diagnostics (IVD) mode assay definition file.

According to an exemplary embodiment, there is provided a local server system comprising a memory and a processor configured to execute instructions, which, when executed by the processor, cause the local server system to perform a method, comprising: receiving, at the local server system, an assay definition file from a server of a cloud computing and storage system, wherein the assay definition file includes code modules for configuring an assay; storing the code modules in the memory of the local server system; receiving, at the local server system, sequencing data from a sequencing device, the sequencing data produced by the sequencing device during a sequencing run for the assay; and applying an analysis pipeline for the assay to the sequencing data, wherein the analysis pipeline includes analysis steps executed by the processor of the local server system in accordance with the code modules from the assay definition file to produce assay analysis results. The code modules for the analysis pipeline may include a code module for a base calling step, the base calling step producing sequence reads. The code modules for the analysis pipeline include a code module for an alignment step, the alignment step producing aligned sequence reads. The code modules for the analysis pipeline include a code module for a variant calling step, the variant calling step applied to the aligned sequence reads to produce variant call results. The server system may further comprise a variome database for storing the variant call results. The method may further comprise displaying the assay analysis results, wherein the display includes an image file for results presentation for the assay. The assay definition file may include the image file for the results presentation for the assay. The assay definition file may include a reference genome file. The assay definition file may include a list of annotation sources. The analysis pipeline may be applied in parallel to the sequencing data corresponding to multiple lanes of a sequencing chip installed in the sequencing device. Each lane of the multiple lanes may correspond to a respective assay, wherein the step of applying an analysis pipeline applies the analysis steps for the respective assay to the sequencing data for the lane. The method may further comprise displaying a page at a user interface of the local server system to a user for selection of the assay definition file for import to the local server system from the cloud computing and storage system. The local server system may further comprise a plurality of assay definition files, wherein the plurality of assay definition files includes a research use only (RUO) mode assay definition file and an in vitro diagnostics (IVD) mode assay definition file. The local server system may further comprise a first database and a second database, wherein the first database stores information for a research use only (RUO) mode of operation and the second database stores information for an in vitro diagnostics (IVD) mode of operation.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Hash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing and storage system. In some embodiments, one or more users can access the computers, or servers, of the cloud computing and storage system over an intranet and/or the Internet. In some embodiments, a user may remotely access the cloud computing and storage system servers through a web client.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Pascal, Basic, Fortran, Cobol, Perl, Python, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

While preferred embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising:
receiving, at a local server system, an assay definition file from a server of a cloud computing and storage system, wherein the assay definition file includes code modules for configuring an assay;
storing the code modules in a memory of the local server system;
receiving, at the local server system, sequencing data from a sequencing device, the sequencing data produced by the sequencing device during a sequencing run for the assay; and
applying an analysis pipeline for the assay to the sequencing data, wherein the analysis pipeline includes analysis steps executed by a processor of the local server system in accordance with the code modules from the assay definition file to produce assay analysis results, wherein the code modules include binary codes executable by the processer for the analysis steps.

2. The method of claim 1, wherein the code modules include a code module for a base calling step, the base calling step producing sequence reads.

3. The method of claim 2, wherein the code modules include a code module for an alignment step, the alignment step producing aligned sequence reads.

4. The method of claim 3, wherein the code modules include a code module for a variant calling step, the variant calling step applied to the aligned sequence reads to produce variant call results.

5. The method of claim 4, further comprising storing the variant call results in a variome database of the local server system.

6. The method of claim 1, further comprising displaying the assay analysis results, wherein the display includes an image file for a presentation of the assay analysis results.

7. The method of claim 6, wherein the assay definition file includes the image file for the results presentation for the assay.

8. The method of claim 1, wherein the assay definition file includes a reference genome file.

9. The method of claim 1, wherein the assay definition file includes a list of annotation sources.

10. The method of claim 1, wherein the analysis pipeline is applied in parallel to the sequencing data corresponding to multiple lanes of a sequencing chip installed in the sequencing device.

11. The method of claim 10, wherein each lane of the multiple lanes corresponds to a respective assay, wherein the step of applying an analysis pipeline applies the analysis steps for the respective assay to the sequencing data for the lane.

12. The method of claim 1, further comprising displaying a page at a user interface of the local server system to a user for selection of a particular assay definition file for import to the local server system from the cloud computing and storage system.

13. The method of claim 1, further comprising a plurality of assay definition files, wherein the plurality of assay definition files includes a research use only (RUO) mode assay definition file and an in vitro diagnostics (IVD) mode assay definition file.

14. A local server system comprising:
a memory; and
a processor configured to execute instructions, which, when executed by the processor, cause the local server system to perform a method, comprising:
receiving, at the local server system, an assay definition file from a server of a cloud computing and storage system, wherein the assay definition file includes code modules for configuring an assay;
storing the code modules in the memory of the local server system;
receiving, at the local server system, sequencing data from a sequencing device, the sequencing data produced by the sequencing device during a sequencing run for the assay; and
applying an analysis pipeline for the assay to the sequencing data, wherein the analysis pipeline includes analysis steps executed by the processor of the local server system in accordance with the code modules from the assay definition file to produce assay analysis results, wherein the code modules include binary codes executable by the processer for the analysis steps.

15. The local server system of claim 14, wherein the code modules include a code module for a base calling step, the base calling step producing sequence reads.

16. The local server system of claim 15, wherein the code modules include a code module for an alignment step, the alignment step producing aligned sequence reads.

17. The local server system of claim 16, wherein the code modules include a code module for a variant calling step, the variant calling step applied to the aligned sequence reads to produce variant call results.

18. The local server system of claim 17, further comprising a variome database for storing the variant call results.

19. The local server system of claim 14, further comprising a plurality of assay definition files, wherein the plurality of assay definition files includes a research use only (RUO) mode assay definition file and an in vitro diagnostics (IVD) mode assay definition file.

20. The local server system of claim 14, further comprising a first database and a second database, wherein the first database stores information for a research use only (RUO) mode of operation and the second database stores information for an in vitro diagnostics (IVD) mode of operation.
